(19) United States
(12) Patent Application Publication
(10) Pub. No.: US 2010/0021400 A1
(43) Pub. Date: Jan. 28, 2010

(54) Active Sunscreen Composition

(75) Inventors: Gregory D. Jay, Norfolk, MA (US); Gregory Crawford, Granger, IN (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Rhode Island Hospital, Providence, RI (US)

(21) Appl. No.: 12/430,739

(22) Filed: Apr. 27, 2009

Related U.S. Application Data
(60) Provisional application No. 61/125,526, filed on Apr. 25, 2008.

(51) Int. Cl. A61K 8/18; A61Q 17/04

(52) U.S. Cl. 424/59

(57) ABSTRACT

The invention relates to topical compositions comprising a fluorescent dye that absorbs UVA and UVB radiation and converts it into a healing light, in combination with a dermatologically acceptable excipient. The compositions of the invention are useful for repairing photo-damaged skin cells and reducing or preventing future skin damage as a result of exposure to UV light.

### REFERENCE TO RELATED APPLICATIONS

This non-provisional patent application claims priority under 35 U.S.C. § 119(e) to U.S. Provisional Application No. 61/125,526, filed Apr. 25, 2008, which is herein incorporated by reference in its entirety.

### FIELD OF THE INVENTION

The present invention relates to compositions and methods for repairing skin damage as a result of exposure to UV radiation.

### BACKGROUND OF THE INVENTION

Of all the cancer risks that abound in the modem world, sunlight is the most difficult to avoid. Many try to shield themselves from harmful UV radiation by coating their skin in compounds like titanium dioxide to scatter and reflect it or cinnamates to absorb the most damaging UVB wavelengths. The problem is they both provide is spotty protection and depend on regular reapplication that many users forget. Combining both type of compounds increases the level of protection, measured by the SPF scale, but, even when used ideally, some radiation can still get through to cause damage. In addition, the commonly referred to SPF scale only applies to UVB radiation, leaving out the UVA and UVC ranges.

The number of skin cancer cases continues to increase in the United States. More than 1 million cases of basal cell or squamous cell cancer will be diagnosed annually. In 2002 alone, the most serious form of skin cancer, malignant melanoma, was diagnosed in nearly 45,000 persons and approximately 7,500 men and women died of the disease. New cases of skin cancer have increased by 100% since 1944, the year when the first effective sunscreen lotion was sold. Since 1981, the incidence of melanoma has increased by nearly 3% per year.

Melanoma is the most common cancer among people ages 25 to 29. The three primary types of skin cancer are basal cell carcinoma, squamous cell carcinoma, and melanoma. Basal cell and squamous cell carcinomas can cause significant sickness. If untreated, basal and squamous cell carcinomas can cause considerable damage and disfigurement. Malignant melanoma on the other hand causes more than 75% of all skin cancer-related deaths. This disease is aggressive and can spread to other organs, most commonly the lungs and liver. Malignant melanoma diagnosed at an early stage usually can be cured, but melanoma diagnosed at a late stage is more likely to progress and cause death.

### SUMMARY OF THE INVENTION

The invention features an “active” sunscreen composition that not only prevents DNA damage from occurring, but induces DNA repair. The composition of the invention is useful for undoing or repairing DNA damage in skin that has occurred in the past, i.e., before its first use, and for reducing or preventing further DNA damage to skin as a result of exposure to UV radiation. The composition described herein absorbs UV radiation from the sun and converts the UV light into skin-healing light of the correct spectral signature. The composition of the invention mediates blue-light-induced DNA repair and is more effective than conventional sunblocks. The composition of the invention may optionally may be incorporated into conventional sunscreen products.

The compositions of the invention are topically applicable and contain a fluorescent dye that absorbs UVA and UVB radiation and converts it the UV light into a visible light in the blue and blue-violet wavelength. For example, the fluorescent dye absorbs radiation in the range of 290-400 nm and emits radiation in the range of 400-500 nm, preferably 415-420 nm. The distance of emitted radiation is relatively narrow (hence, the term nano-“laser”), e.g., the distance of emitted radiation ranges from 0.1 mm to 10 mm, preferably 0.5 mm to 10 mm, more preferably 5 mm to 7 mm. For example, the distance of the emitted radiation is 5 mm.

The fluorescent dye is provided in an amount effective to induce or activate a DNA repair enzyme in a skin cell. For example, the compositions comprise a fluorescent dye in a 0.1% to 10% solution, e.g., 1%-2%, which is applied to skin at least once a day in an amount ranging from 0.01%/cm² to 5%/cm², preferably 0.01%/cm² to 2%/cm², more preferably 0.01%/cm² to 1%/cm , even more preferably 0.01 %/cm² to 0. 5%/cm².

Suitable fluorescent dyes for use in the composition of the invention include organic compounds such as stilbene (C₂₈H₂₀O₆S₂.2Na) (e.g., stilbene-420; 2,2″-([1,1′-biphenyl]-4.4′-dyldi-2,1-ethenediyl)bis-benzenesulfonic acid disodium salt; Stilbene3; Catalog No.: 04200, CAS No.: 27344-41-8, Exciton) and derivatives thereof such as 4,4′-diaminostilbene, and bistriazinyl derivatives; derivatives of benzene and biphenyl, e.g., styryl derivatives; pyrazolines; derivatives of bis(benzoxazol-2-yl); coumarins; carbostyrils; naphthalimides; s-triazines; pyridotriazoles and inorganic fluorescent glasses. For example, Stilbene-420, Tinopal CBS-X (a distyryl biphenyl derivative), Keyflour White (an oxazole) and Lumilass B (an inorganic fluorescent glass) may be used in the composition of the invention.

An effective amount is an amount of a compound (e.g., one or more fluorescent dyes, alone or in a combination), required to induce, reduce or prevent the DNA damage as a result of exposure of cells to UV light.

Optionally, the composition of the invention further includes a nanolaser particle, such as is a nano-sized lasing fibril created from reactive liquid crystal materials (e.g., cholestric liquid chrystal). The use of a nanolaser particle in combination with one or more fluorescent dyes as described herein allows the dye's re-emission spectrum to be narrowed and thus improves the efficiency of the dye's ability to absorb and convert UV light into visible blue and blue-violet light.

The composition is formulated into a delivery system such as a cream, shampoo, gel, lotion, soap, oil, stick or spray and is useful for reversing, reducing or preventing UV-induced DNA damage in a skin cell by topically applying the composition to skin prior to or during exposure to UV light.

The composition of the invention can also be used in a regimen for photoprotecting or reversing UV damage of the skin, the lips, the nails, the hair, the eyelashes, the eyebrows, and/or the scalp by topically applying the composition in a topically applicable, cosmetically or dermatologically acceptable excipient daily, or when UV-exposure is anticipated For example, the composition is applied at least once daily. Optionally, the composition further comprises at least one compound selected from the group consisting of anti-oxidants, sunscreens, moisturizers, bleaching agents, depigmentation agents, darkening agents, surfactants, foaming agents, conditioners, humectants, fragrances, anti-aging agents, anti-inflammatory agents, and anti-cancer agents. The composition induces an increase in an amount or activity of a DNA repair enzyme (e.g., photolyase) of said skin cell.

Other features and advantages of the invention will be apparent from the following description of the preferred embodiments thereof, and from the claims. References cited are hereby incorporated by reference.

### BRIEF DESCRIPTION OF THE DRAWINGS

FIGS. 1a.-d. are diagrams of steps in the fabrication process of nano-sized chiral fibers: heat material (a); capillary fill (b); NaOH etch (c); and liberated (d).

FIGS. 2a.-c. are photomicrographs of dermal fibroblasts exposed to UV radiation in the presence and absence of a UV protective composition: (A ) stilbene, (B) commercially available sunblock or (C) nothing.

FIG. 3 is a series of digitized images depicting the extent of DNA damage by Comet Analysis in dermal fibroblasts exposed to UV radiation while protected by either (A ) stilbene, (B) commercially available sunblock or (C) nothing; Images were processed in MatLab and thresholds were set to detect comet tails which simple visual inspection may not see.

FIG. 4a is an illustration of the molecular profile within nano-sized chiral fibers.

FIG. 4b is a photograph of an SEM image of nanofibrils.

FIG. 4c is a line graph showing nanofibril output and dye fluorescence.

### DETAILED DESCRIPTION

Some DNA repair mechanisms in skin cells are photosensitive to light at approximately 415-420 nm, just above the wavelength for ultraviolet radiation. Activity of these mechanisms is also suspected to be proportional to the intensity of exposure. By converting a portion of incoming UV radiation into this beneficial wavelength, any damage from UV rays that get through is repaired.

### UV Radiation and Cancer

UV radiation is a potent carcinogen to the human skin. The origin of sun-induced skin cancers is believed to be traced back to DNA damage—a photo-induced mechanism which harms the DNA in the skin. Repair of DNA lesions is thought to be the primary defense mechanism against oncogenic damages.

### DNA Damage and Repair

UVA and UVB light (ultraviolet radiation, 290 nm≦λ_{(UVB)}≦320 nm, and 320 nm≦λ_{(UVA)}≦400 nm) damage the skin. In some cases, this damage would be repaired or reversed with a certain spectral irradiation in the visible spectrum. UV radiation exposure is and has always been a risk factor for land-dwelling animals. The high-energy photons are capable of penetrating cell membranes and transferring their energy to atoms, creating free radicals that damage DNA or alter its structure. Various organisms have developed their own method for dealing with this damage. The basic mechanism generally involves recognizing damage by looking for mismatched base pairs. If a single base is at fault, it is simply removed and replaced using DNA ligase. Such errors are usually caused by mistakes in DNA replication and can be dealt with by the DNA polymerase itself. If a larger sequence needs to be repaired, more complex mechanisms are necessary, but they follow the same basic principle or removing the error, checking a reference, and replacing the erroneous sequence with the correct one.

One particularly serious type of DNA damage is the formation of thymine dimers, which warp the basic double-helix of the DNA strand, causing serious problems during replication. This type of error is one of the more common results of UV radiation. They cause major problems in replication if not fixed. Bacteria and other prokaryotes use a type of enzyme known as a photolyase to break these accidental bonds, a type of direct reversal. Typically, these photolyases are activated by visible light. Unfortunately, evidence of this mechanism in higher organisms has been limited, especially in placental mammals. Related sequences exist, such as cryptochrome, but their function as a DNA repair mechanism is not well explored.

DNA repair mechanisms are crucial to the continued function of living cells. A light-activated mechanism for DNA repair allows for quick repair of dimmers before they can become permanent. People today badly abuse their skin by exposing it to intensities and durations it has not evolved to handle. In particular, many seek to over stimulate another UV protection mechanism, tanning, to produce a cosmetically pleasing effect. As a result, despite their effectiveness, repair mechanisms cannot keep up and errors begin to build. If done in small doses, one may be able to get away with it but the hours on end in the sun with little to no break or regular reapplication of sunblock result in a great deal of damage, as seen in sunburns. Having a mechanism that activates in response to visible light striking the cell would be more efficient than leaving it constantly active, especially of the light is on the lower end of the visible spectrum which one is unlikely to encounter in nature without also encountering UV with it.

### Conventional Sunscreens and Skin Cancer

In the United States alone, there are more than one-million cases of non-melanoma skin cancer diagnosed every year. Melanoma skin cancer represents 4% of all skin cancers, which may seem small, but unfortunately accounts for more than 75% of all skin cancer related deaths. It is in this regard that health experts and medical practitioners alike are adamant about protecting our skin against the sun. Less serious outcomes of sun irradiation are photo-aging of the skin. Excessive exposure to the sun early in life can make a person look older than he/she really is, resulting in premature wrinkling. Photo-aging, unlike natural aging, results in course, dry skin, freckling, skin discoloration, leathery skin, and deep wrinkles.

Conventional sunscreens fall into two general categories organic and inorganic. Inorganic sunscreens like zinc oxide (ZnO) and titanium oxide (TiO₂) tend to absorb UV light to form radical species or simply scatter the incoming light. These substances are usually seen as a thick white paste, but, for cosmetic reasons, the particles are ground to a microscopic size and suspended in oils to make them more of a transparent lotion. Without this adjustment, they can block out about ninety-nine percent of visible and ultraviolet light and function as a “sunblock.” When made more cosmetically pleasing, the particles decrease in their effectiveness to about ninety-four to ninety-seven percent of UVB being blocked. Organic sunscreens, on the other hand, are almost entirely based on absorbing the energy of UV wavelengths using aromatic compounds. While effective, each molecule can only absorb the one photon before becoming inactive. In spite of these drawbacks, both of these categories are effective to a certain extent. However, they require regular re-application as they tend to wash off with the sweat generally associated with hot sunny days. In fact, they require far more regular reapplication than most of their users are willing to perform. Worse still, as they are made transparent for cosmetic reasons, it is difficult to tell if one has covered all exposed skin.

UVB isn't the only type of ultraviolet radiation. In fact, ultraviolet radiation comes in 3 types: A, B and C. UVB and C are the most dangerous, having wavelengths in the 200-300 nm range, giving them better penetrating power. Yet, UVA can still be damaging and is thought to be the primary source of photoaging effects. The standard Sun Protection Factor (SPF) rating system only takes the protection against UVB into account. The SPF rating is based on the ratio of UV exposure needed to cause minimal erythema to protect skin to the same level of erythema on unprotected skin. Basically, it is a ratio of how much UV exposure is necessary to damage sunblock-protected skin to how much is necessary to damage unprotected skin. Some sunblocks and sunscreens claim some protection against UVA but no scale is uniformly accepted yet. UVA protection, in general, is just ten percent of the UVB protection. Anthranilate and avobenzone are exemplary ingredients for blocking out UVA wavelengths.

UVC, for the most part, is not stopped well by most sunscreens and can damage skin severely. It is, however, mostly absorbed by the ozone layer. On the other hand, human-created sources used for germicidal purposes are quite common. Designed to kill bacteria and other microbes by destroying their DNA, such devices can cause similar damage to human skin with prolonged exposure. In addition, repeated exposure to even small doses over a prolonged time period can be harmful.

### Active Sunscreen Compositions of the Invention

The invention provides a composition comprising a fluorescent dye which can absorb UVA and UVB light and emit narrow band wavelengths in the visible spectrum (blue and blue-violet light) which induces or activates DNA repair enzymes in skin cells, such as photolyases. As such, the compositions provide an “active” defense against UV radiation-induced skin damage (i.e., converts damaging UV radiation into healing light which repairs, reduces and prevents further UV-induced DNA damage to skin), in contrast to conventional sunscreens which only passively block UV radiation.

Fluorescent dyes suitable for use in the composition of the invention are those that absorb radiation in the range of 290-400 nm (i.e., the range of UVA and UVB radiation) and emit radiation in the range of 400-500 nm, preferably 415-420 nm, which is ideal for activating DNA repairs enzymes, such as photolyase, in skin cells. In order to activate DNA repair enzymes, the emitted radiation must be able to penetrate the epidermis. The thickness of the human epidermis various throughout the body. For example, the layer of epidermis on human eyelids ranges from 0.1 mm to 0.5 mm, whereas the epidermis may be as thick as 10 mm at the absolute thickest point. Thus the fluorescent dye suitable for use in the composition of the invention must be capable of emitting radiation at a distance which penetrates the human epidermis at it's thinnest and thickest point. For example, the distance of emitted radiation ranges from 0.1 mm to 10 mm, preferably 0.5 mm to 10 mm, more preferably 5 mm to 7 mm, even more preferably 5 mm.

Suitable fluorescent dyes for use in the composition of the invention include organic compounds such as stilbene (C₂₈H₂₀O₆S₂.2Na) (e.g., stilbene-420; 2,2″-([1,1′-biphenyl]-4.4′-dyldi-2,1-ethenediyl)bis-benzenesulfonic acid disodium salt; Stilbene3; Catalog No.: 04200, CAS No.: 27344-41-8, Exciton) and derivatives thereof such as 4,4′-diaminostilbene, and bistriazinyl derivatives; derivatives of benzene and biphenyl, e.g., styryl derivatives; pyrazolines; derivatives of bis(benzoxazol-2-yl); coumarins; carbostyrils; naphthalimides; s-triazines; pyridotriazoles and inorganic fluorescent glasses. For example, Stilbene-420, Tinopal CBS-X (a distyryl biphenyl derivative), Keyflour White (an oxazole) and Lumilass B (an inorganic fluorescent glass) may be used in the composition of the invention. In a particular embodiment, the composition of the invention comprises Stilbene-420. Other stilbene derivatives are described in Klatzkin et al., 1948, Biochem. J. 42(3):420-424.

The fluorescent dye is provided in an amount effective to induce or activate a DNA repair enzyme in a skin cell. For example, the compositions comprise a fluorescent dye in a 0.1% to 10% solution, e.g., 1%-2%, which is applied to skin at least once a day in an amount ranging from 0.01%/cm² to 5%/cm², preferably 0.01%/cm² to 2%/cm², more preferably 0.01%/cm² to 1%/cm², even more preferably 0.01%/cm² to 0.5%/cm². The concentration of dye accumulates in the skin after multiple applications to provide continued protection against damaging UV radiation.

### Nanolaser Based Active Sunscreen Compositions of the Invention

The inventions provides compositions and methods for sun-protection based on the use of fluorescent dyes to convert damaging UV wavelengths into blue light to induce DNA repair. In one embodiment, the active sunscreen composition described herein can be combined with cholesteric liquid crystal technology to form nanolaser particles. The properties of nanolaser liquid crystals improve the efficiency of the conversion of UV light into a healing blue light. Thus, nanolaser particles may be combined with one or more fluorescent dyes described herein and formulated into a self-healing sunscreen lotion (i.e., “active” sunblock).

The fluorescent dyes described herein, while less efficient at absorbing and re-emitting light, do still produce the same sort of conditions as when combined with a nanolaser particle. However, the tunable nature of nanolasers allow the dye's re-remission spectrum to be narrowed while also increasing the efficiency of its absorption and conversion of UV wavelengths. This enhancement stimulates the photorepair mechanism to its maximum extent. By “tuning” the frequencies, the photo-repair effect is optimized for the lowest concentration of the fluorescent dye/nanolaser. Mixing nanolaser particles with one or more dyes is helpful to narrow the range down. Mixing nanolasers with more than one dye can help extend the range of absorption for the nanolaser/dye composition if desired. The term ‘nanolaser’ is used to describe the particles since they emit narrow bandwidth of light; however, for the proposed application, the emitted light is not strictly speaking a laser but rather a relatively narrow emission line.

A template-based approach can be used to create fibrils on the nanoscale (R<100 nm), which possess chiral symmetry. The pitch of the chirality, or periodicity, is commensurate with the wavelength of visible light. By incorporating a laser dye in the fibril, the periodicity introduces feedback, much like a laser cavity; therefore the bandwidth (full width at half maximum) of the fluorescent output of the fibril is very narrow when optically irradiated. Furthermore, this output is tuned to a specific portion of the visible spectrum. It has been shown that blue light leads to the repair of sun-damaged skin. By tuning the peak position and width of the fibril nanolaser output, damaging UVA and UVB rays are completely absorbed and converted to skin healing blue light.

Nanolasers are made from liquid crystals, utilizing their photonic band gap properties to produce a mini-laser diode. Due to their size and structure, their own periodicity can be very similar to that of visible light. Furthermore, their structure is manipulated with a variety of methods including surface alignment techniques, external electric fields and finite crystal geometries known in the art. By doping the crystals with appropriate fluorescent dyes, a functional lasing device is made that works off of an ambient light source, like sunlight. One medium for making nanolasers is the cholesteric liquid crystal, which exhibits periodicity in only one dimension with its chiral molecular symmetry. By adjusting the crystal structure and concentration of dye, a nanolaser crystal is “tuned” to more efficiently re-emit light and tighten the emission spectrum. Thus, liquid crystal nanolasers narrow down the emission range of the dyes and induce even higher rates of DNA repair in skin cells, even to the point of correcting damage accumulated from before application.

An exemplary medium for lasing from liquid crystal based PBG materials is the cholesteric liquid crystal, which exhibits periodicity in one dimension due to chiral molecular symmetry. These particles or nano-lasers reflect normally incident light, λₒ, according to the Bragg condition, λₒ=n_{A}P, where n_{A} is the average index of refraction of the liquid crystal (0.1≦n_{A}≦0.3) and P is the pitch of the cholesteric liquid crystal. The bandwidth of the cholesteric liquid crystal, Δλ, is given by Δλ=λΔ=λₒΔn where, An is the birefringence (0.1≦Δn≦0.3). Due to the chiral symmetry, a right handed chiral pitch reflects right-handed circularly polarized light and transmits left-handed circularly polarized light. The chiral material can exhibit pitch lengths that reflect UV through near-IR wavelength (250 nm≦λₒ≦2 μm).

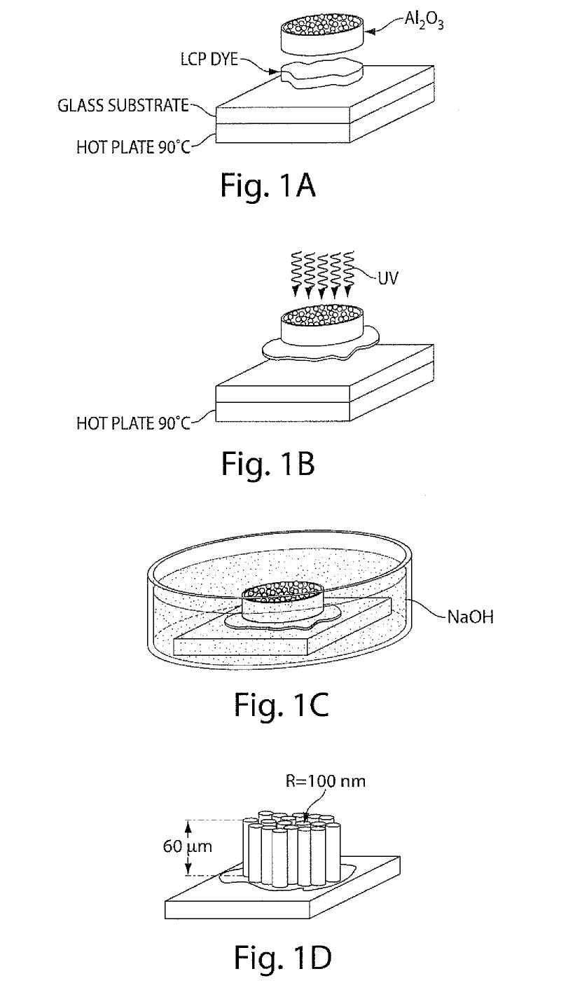

Nano-sized lasing fibrils are created from reactive liquid crystal materials. Reactive liquid crystals, sometimes referred to as reactive mesogens, are those materials which are initially low molecular weight so they can be easily manipulated with surfaces, external fields, and confinement much like liquid crystal materials found in laptop computers; however, after the desired ordering and alignment is achieved, they can be photo-polymerized to indefinitely capture the molecular configuration. In order to enable lasing, chiral nano-fibrils are doped with a laser dye, such as the fluorescent dyes described herein. The reactive chiral liquid crystal doped with laser dye is initially be filled in an Al₂O₃ template with 100 nm radii channels at a temperature where the cholesteric liquid crystal phase is stable (FIG. 1a). The compositions exhibit nematic liquid crystal phases at temperatures in the range 80° C.≦T≦125° C. Since the channel radius of the template is smaller than the desired pitch of the reactive chiral liquid crystals, the pitch axis of the material is designed to be parallel to the cylindrical axis of the channel much like the bulk cholesteric liquid crystal phase. The chiral symmetry is then be captured with photo-polymerization (FIG. 1b), and the fibrils are liberated from the Al₂O₃ template through a NaOH etching process (FIG. 1c) leaving behind an ordered array of chiral fibers (FIG. 1d). The templates are made with channel sizes of 1-100 nm radii, e.g., with radii of ˜25 nm.

### Uses for Active Sunscreen Compositions of the Invention

Applications for the active sunscreen compositions of the invention are wide and varied. The compositions prevent the everyday damage people receive from not wearing enough sunblock, but also be able to repair the damage they have already received. In addition, the protection is also effective against UVA wavelengths which most commercial sunblocks only offer limited effect on and provide a method to prevent the photoaging caused by those wavelengths. Medical applications include treatment of people with family histories of skin cancer or medical conditions like xeroderma pigmentosum. By carefully choosing the range of dyes used, a broader range of wavelengths can be protected against.

Another application of the technology is the protection of astronauts on long-term missions. Long-term exposure to radiation during space travel is a major limiting factor to duration of missions, and one whose risks are not yet fully explored. Daily treatments are used to reverse DNA damage by exposure to otherwise damaging wavelengths of light. The active sunblock uses the same damaging radiation as power or to enhance the aforementioned daily treatments. Using part of the spectrum from both natural and artificial light sources allows them to keep pace with the damage as it is initiated.

### Formulations

Formulations suitable for topical administration include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the drug with a dermatologically acceptable carrier such as a lotion, cream, ointment or soap. Useful are carriers capable of forming a film or layer over the skin to localize application. Additionally, the carrier for a topical formulation can be in the form of a hydroalcoholic system (e.g quids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems. Nonlimiting examples of the topical carrier systems useful in the present invention are described in the following references: “Sun Products Formulary”, Cosmetics & Toiletries, vol. 105, pp. 122-139 (December 1990); “Sun Products Formulary”, Cosmetics & Toiletries, vol. 102, pp. 117-136 (March 1987); U.S. Pat. No. 4,960,764; and U.S. Pat. No. 4,254,105.

An optional component of the compositions useful is at least one humectant/moisturizer/skin conditioner. A variety of these materials can be employed and each can be present at a level of from about 0.1% to about 20%, alternatively from about 1% to about 10% and yet alternatively from about 2% to about 5%. These materials include urea; guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel ); polyhydroxy alcohols such as sorbitol, glycerol, hexanetriol, propylene glycol, hexylene glycol and the like; polyethylene glycol; sugars and starches; sugar and starch derivatives (e.g., alkoxylated glucose); hyaluronic acid; lactamide monoethanolamine; acetamide monoethanolamine; and mixtures thereof. Humectants/moisturizers/skin conditioners useful herein are the C3 -C6 diols and triols, and also aloe vera gel. Especially preferred is the triol, glycerol, and also aloe vera gel.

The compositions described herein are optionally added to conventional sunscreens A wide variety of one or more sun screening agents are suitable for use in the present invention and are described in U.S. Pat. No. 5,087,445; U.S. Pat. No. 5,073,372; U.S. Pat. No. 5,073,371; and Segarin, et al., at Chapter VIII, pages 189 et seq., of Cosmetics Science and Technology.

Certain useful in the compositions of the instant invention ethylhexyl p-methoxycinnamate, octocrylene, octyl salicylate, oxybenzone, or mixtures thereof. Other useful sunscreens include the solid physical sunblocks such as titanium dioxide (micronized titanium dioxide, 0.03 microns), zinc oxide, silica, iron oxide and the like. Without being limited by theory, it is believed that these inorganic materials provide a sun screening benefit through reflecting, scattering, and absorbing harmful UV, visible, and infrared radiation.

Still other useful sunscreens are those disclosed in U.S. Pat. No. 4,937,370; and U.S. Pat. No. 4,999,186. The sun screening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range. These sun screening agents provide higher efficacy, broader UV absorption, lower skin penetration and longer lasting efficacy relative to conventional sunscreens.

Each publication and patent document cited herein is incorporated herein by reference in its entirety as if each such publication or document was specifically and individually indicated to be incorporated herein by reference. Citation of publications and patent documents is not an admission that any is pertinent prior art, nor does it constitute any admission as to the contents or date of the same.

### EXAMPLES

### Example 1

### Induction of DNA Repair in Skin Cells by Fluorescent Dye Stilbene-420

Using a model for human skin (confluent human dermal fibroblasts, Lonza CC-2511), a fluorescent dye was used to evaluate induction of DNA repair. Stilbene-420 was selected for having a broad range of absorption in the UVB and UVA ranges while retaining a relatively narrow emission spectrum centered around 420 nm. A 2% solution of stilbene in water was used for this study.

Stilbene-420 was compared to both unshielded cells and commercial SPF 45 sunblock in their ability to protect Normal Human Dermal Fibroblast cells raised on Lonza FGM-2 media from exposure to the full power of a solar simulator. A CometAssay was used to assess DNA damage to the cells. The resulting data was filtered and analyzed with an Integral of Student's Probability Density.

Using Fibroblast Grown Medium with 10% FBS, normal Human Dermal Fibroblasts (obtained from Lonza Clonetics) were grown to confluence in a 12-well plate. After 6 days, one of the wells was harvested by scraping and stored at 4° C. Using custom aluminum holders, a UV-transparent quartz window was placed above each well, and space between the window and holder sealed with 5% agarose. The windows were then covered with 0.5mL of 0.5% Stilbene 420 florescent dye in water, commercial SPF 45 sunblock or left alone. Then, the plate was exposed to a Solar Simulator for 22.5 minutes at full intensity. Afterwards, the cells were harvested by scraping and all the cells were suspended in low-melting point for the CometAssay using a kit produced by Trevigen. Though the results of DNA damage are often easy to see, for example, cell death, quantifying the extent to which this damage has occurred can be difficult. The CometAssay is a test measures fairly low levels of DNA Damage. The basic concept of the test is that, given more incipient DNA damage that is dealt, the more fragments of DNA are created and the smaller they become. By embedding the cells in a low melting point agarose gel and lysing them, release of these DNA fragments occurs without inducing further damage. A simple gel electrophoresis process causes the fragments to begin to migrate out of the lysed cells. As smaller fragments move faster, a comet-like tail develops, which can be visualized by using a dye like SYBR1 Green. The relative size, intensity and length of this tail are directly related to the amount of DNA damage a cell has received.

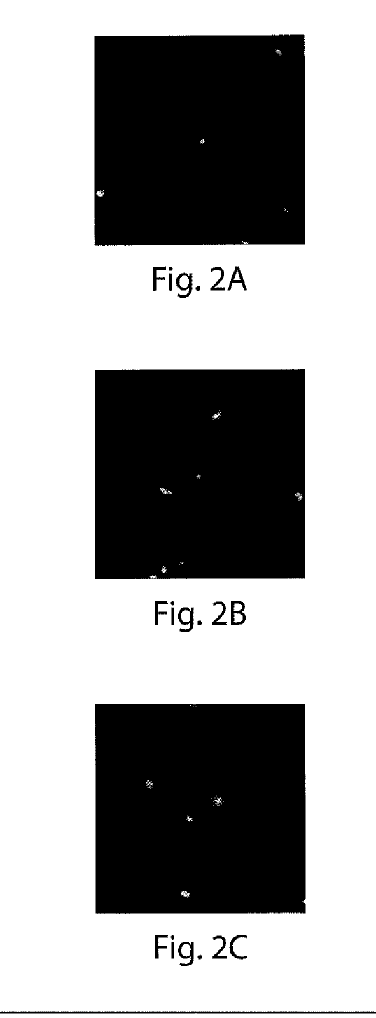

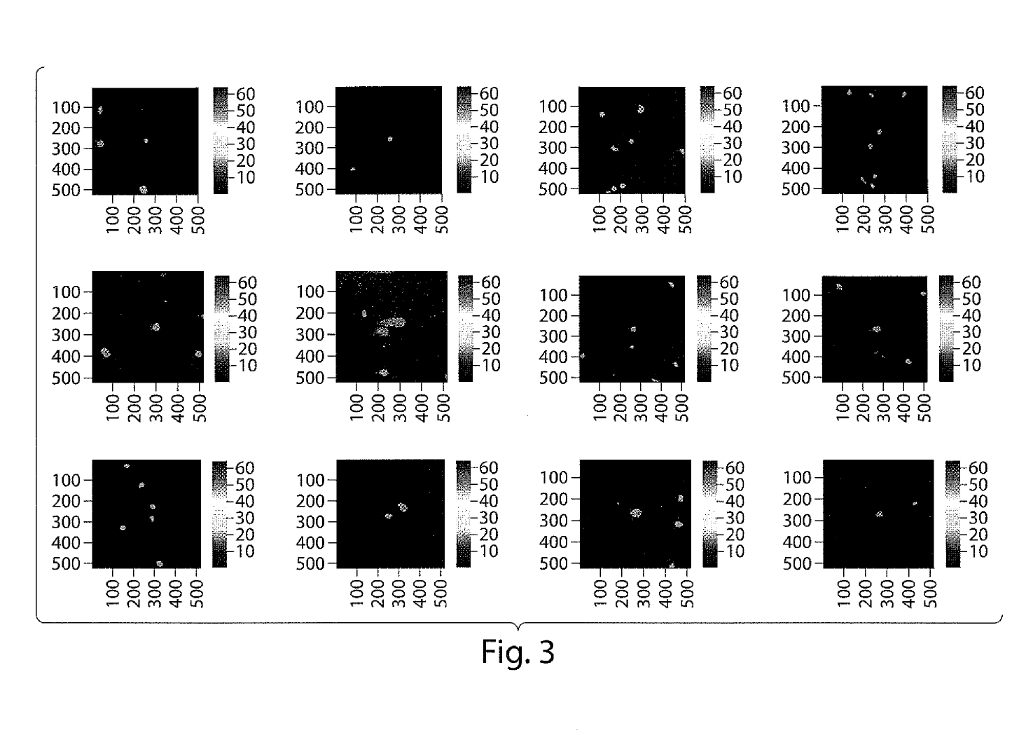

After lysing the cells and running them on an electrophoresis apparatus for 25 minutes at 20 volts, the DNA fragment “tails” were visualized with SYBR I Green dye under a microscope using a 492 nm laser and stored (FIGS. 2a.-c.). Using a MatLAB script, the images were filtered by their intensity to isolate individual cells (FIG. 3). As shown in FIG. 2, unprotected cells take on a comet-like appearance by forming a tail in the direction of electrophoretic current flow. This is indicative of damaged DNA which forms a heterogeneous population of DNA strand lengths. The stillbene protected cells do not form tails indicative of no damage and the sunblock protected cells appear like ‘commas’ which points to partial damage.

Each cell was measured by the width of the “core,” where the larger, less-damaged pieces of DNA remained, and the length of the “tail” produced from the migration of the smaller fragments. These values were compared in several ratios and a Student t-test was performed (Table 1). From those t-values an Integral of Student's Probability Density was calculated assuming an unequal variance between the different protections. This latter value determines the probability that the data comes from the same population as the control data. For the sunblock-protected cells, there is just an 11% chance when comparing the ratio of tail length to total length of the “comet” to the same ratio for the control. For the unprotected cells, the probability drops to just 4.7%. But, for the dye-protected cells, there was a 96.6% came from the same population, indicating the dye provided the best protection, keeping the cells closest to their original state.

TABLE 1
Comet Analysis Tail Sizes of Protected and Unprotected Irradiated Dermal Fibroblasts.

| | | Tail Length/ | Tail Length/ | Integral of Student's | Integral of Student's | Integral of Student's |
|---|---|---|---|---|---|---|
| | Difference | Core Width | Total Length | Probability Density for | Probability Density for | Probability Density for |
| | T-value | T-Value | T-Value | Difference | Tail Length/Core Width | Tail Length/Total Length |
| No | 1.6722 | 2.1999 | 2.3466 | 13.33% | 6.40% | 4.70% |
| Protection | | | | | | |
| Sunblock | 0.9466 | 2.1327 | 1.9420 | 42.64% | 7.31% | 10.89% |
| Protected | | | | | | |
| Stilbene 420 | 0.1870 | 0.2257 | 0.0437 | 85.83% | 82.7% | 96.63% |
| Protected | | | | | | |

From the statistical analysis, the data indicates that this method of protection seems to leave the Stilbene 420 protected cells the closest to the original population. In fact, the population of Stilbene-protected cells has more than a forty percent greater chance of being from the same population of unexposed cells than the sunblock protected cells do. Future applications of this technology range from a new generation of sunblock to keeping astronauts healthy during long trips to other planets.

The data indicated that the stilbene 420 dye is surprisingly effective in protecting the cells exposed to UV light. The amount of DNA damage was more limited with fewer fragments shown in the CometAssay. Even to the naked eye, two cells from different treatments look noticeably different, with the stilbene-protected cells having a narrower, shorter tail.

### Example 2

### Fabrication of Nanolaser Based Active Sunscreen Composition

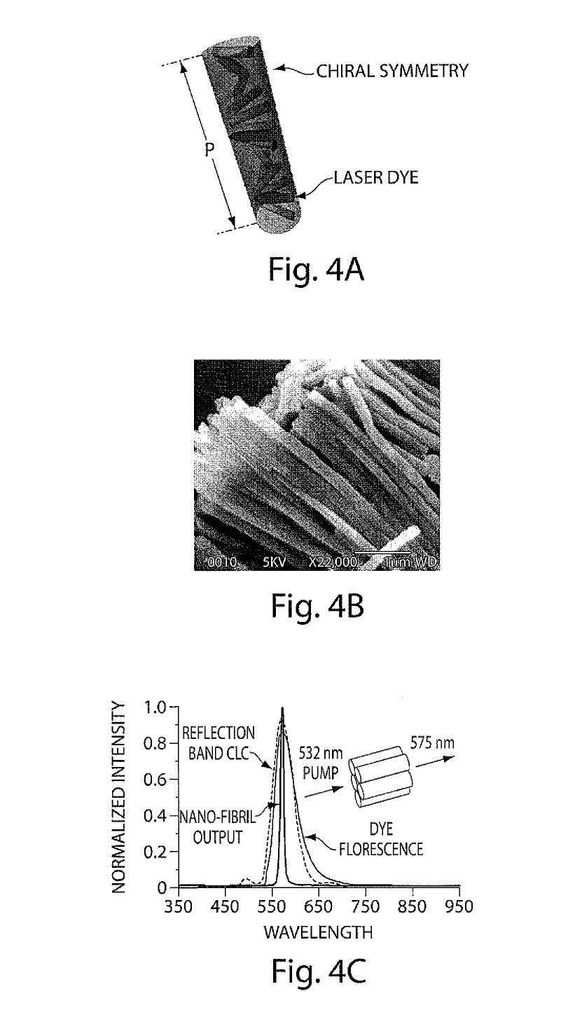

Nano-sized lasing fibrils were fabricated as shown in FIGS. 4a.-c. A reactive diacrylate liquid crystal (RM257 from EM Industries) was employed with a chiral composition to create a reflection band centered at λₒ˜560 nm. The material was doped with 1% of a laser dye (pyrromethene 580 from Exciton) with a fluorescence band centered at ˜550 nm. FIG. 4a shows an illustration of the chiral molecular profile of the nano-fibrils and a corresponding scanning electron micrograph of chiral nano-fibrils prepared (FIG. 4b). Different samples of low and high molecular liquid crystals and dye were made. The sample was irradiated with a frequency doubled Nd:YAG pulsed laser (Quantel) operating at a wavelength of α=532 nm, a repetition rate of 10 Hz and a maximum pulse energy of 200 mJ. FIG. 4c shows the corresponding data for the nano-fibril output and the dye fluorescence band. The reflection band of the liquid crystal (green dashed line) is shown in FIG. 4c demonstrating a peak wavelength λₒ˜560 nm and a bandwidth Δλ˜80 nm. The fluorescence spectrum, (blue line) is also shown for comparison purposes in FIG. 4(c). The output of the nanofibrils is a narrow line (˜5 nm full-width-half-maximum).

### Example 3

### Evaluation of Nanolaser Based Active Sunscreen Composition

Nanolaser based active sunscreen compositions or the invention are evaluated in an animal model. The SKH-1 hairless mouse is a common UVB-induced skin cancer model used to examine effects of anti-carcinogenic substances. Nanolasers based active sunscreen compositions are formulated into an active sunblock lotion, and the mice are contacted with the lotion in an area on their lower spine, which cannot be reached by individuals for cleaning. The mice are then exposed to UV light and are observed for skin lesions and other signs of skin cancers.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A topically applicable composition comprising a fluorescent dye that absorbs radiation in the range of 290-400 nm and emits radiation in the range of 400-500 nm, in an amount sufficient to increase activity of a DNA repair enzyme in a skin cell.

2. The composition of claim 1, wherein said fluorescent dye comprises a stilbene compound.

3. The composition of claim 1, wherein the emitted radiation is 415-420 nm.

4. The composition of claim 1, wherein the distance of the emitted radiation is 0.1 mm to 10 mm.

5. The composition of claim 4, wherein the distance of the emitted radiation is 0.5 mm to 10 mm.

6. The composition of claim 5, wherein the distance of the emitted radiation is 5 mm to 7 mm.

7. The composition of claim 6, wherein the distance of the emitted radiation is 5 mm.

8. The composition of claim 1, wherein the fluorescent dye is selected from the group consisting of derivatives of stilbene and 4,4′-diaminostilbene; derivatives of benzene and biphenyl; pyrazolines; derivatives of bis(benzoxazol-2-yl); coumarins; carbostyrils; naphthalimides; s-triazines; pyridotriazoles and inorganic fluorescent glasses.

9. The composition of claim 8, wherein the fluorescent dye is selected from Stilbene-420, Tinopal CBS-X, Keyflour White, and Lumilass B.

10. The composition of claim 1, further comprising a nanolaser particle.

11. The composition of claim 10, wherein the nanolaser particle is a nano-sized lasing fibril.

12. The composition of claim 11, wherein the nano-sized lasing fibril comprises a cholestric liquid crystal.

13. The composition of claim 1, wherein said composition is formulated into a delivery system comprising creams, shampoos, gels, lotions, soaps, oils, sticks or sprays as a vehicle for topical application.

14. A method for preventing or reversing UV-induced DNA damage in a skin cell, comprising contacting said skin cell with the composition of claim 1 prior to or during exposure to UV light.

15. A regimen for photoprotecting the skin, the lips, the nails, the hair, the eyelashes, the eyebrows, and/or the scalp against the damaging effects of UV-radiation, comprising topically applying the composition of claim 1 in a topically applicable, dermatologically acceptable excipient.

16. The method of claim 14, wherein said composition is applied at least once daily.

17. The method of claim 14, wherein said composition further comprises at least one compound selected from the group consisting of anti-oxidants, sunscreens, moisturizers, bleaching agents, depigmentation agents, darkening agents, surfactants, foaming agents, conditioners, humectants, fragrances, anti-aging agents, anti-inflammatory agents, and anti-cancer agents.
